(19) United States
(12) Patent Application Publication
(10) Pub. No.: US 2026/0224819 A1
(43) Pub. Date: Aug. 6, 2026

(54) INJECTION DEVICE FOR DELIVERING A DRUG

(71) Applicant: SHL Medical AG, Zug (CH)

(72) Inventors: Daniel Carlsson, Nacka Strand (SE); Marcus Söderlund, Nacka Strand (SE)

(21) Appl. No.: 19/146,380

(22) PCT Filed: Dec. 28, 2023
(86) PCT No.: PCT/EP2023/087932

§ 371 Date: Jul. 8, 2025

(30) Foreign Application Priority Data
Jan. 16, 2023 (EP) 23151854.9

(51) Int. Cl. A61M 5/32

(52) U.S. Cl. CPC A61M 5/3234; A61M 5/3221

(57) ABSTRACT

The present disclosure provides an injection device for delivering a drug, extending generally along a longitudinal axis, and having a proximal end configured to point towards a dose delivery site and a distal end pointing away from the dose delivery site. The injection device includes a drug container comprising a barrel and a plunger axially movable inside the barrel, and a safety device. The safety device includes a housing, a drug container holder holding the drug container in the housing, and a needle assembly attached to a proximal end of the drug container holder and comprising a needle. The safety device further includes a manually actuatable plunger assembly comprising a plunger rod capable of proximally pushing the plunger for expelling a dose of the drug through the needle. The safety device further includes a biasing member capable, when released, of moving the needle assembly and the drug container holder from an end of injection position where the needle extends partially out of a proximal part of the housing, to a final retracted lock-out position where the needle extends fully inside the housing. The safety device further includes cooperating elements of the housing and the drug container holder is capable, when engaged, of retaining the drug container holder in the end of injection position, and when disengaged, of releasing the biasing member. The safety device further includes a sheath lock having a leg extending radially between the drug container holder and the housing. and capable, in an end of injection position of the sheath lock, of disengaging the cooperating elements.

### TECHNICAL FIELD

The invention relates to injection devices for delivering a drug.

### BACKGROUND

Injection devices typically fall into two categories-manual devices and autoinjectors. A conventional autoinjector may provide the force for administering the injection by a spring, and a trigger button or other mechanism may be used to activate the injection. On the contrary, in a conventional manual device, a user must provide force to drive a drug through a needle. This is typically done by some form of button/plunger rod that has to be continuously pressed during the injection.

Safety devices are manual injection devices providing needle safety. They are adapted to avoid accidental needle injuries before, during and after an injection of a medication or drug contained in a pre-filled drug container.

Safety devices covering a needle of a pre-filled drug container before and after use are well known. When the needle is covered, the device is said “locked-out”.

Typically, these devices comprise a needle shield used to surround the needle after injection, the needle shield being either manually moved or moved by the action of a biasing member, for instance a pre-compressed spring.

Different type of safety devices known in the state of the provide needle safety by arranging the needle movable relative to a housing, wherein the needle is retracted into the housing after the injection.

It is an object of the present invention to provide a safety device adapted to retract the needle into the housing after the injection.

### SUMMARY

Reference should now be made to the appended claims.

The invention refers to an injection device for delivering a drug, extending generally along a longitudinal axis, and having a proximal end configured to point towards a dose delivery site and a distal end pointing away from the dose delivery site, the injection device comprising:
a drug container comprising a barrel and a plunger axially movable inside the barrel,
a safety device comprising:
   a housing,
   a drug container holder holding the drug container in the housing,
   a needle assembly attached to a proximal end of the drug container holder and comprising a needle,
   a manually actuatable plunger assembly comprising a plunger rod capable of proximally pushing the plunger for expelling a dose of the drug through the needle,
   a biasing member capable, when released, of moving the needle assembly and the drug container holder from an end of injection position where the needle extends partially out of a proximal part of the housing, to a final retracted lock-out position where the needle extends fully inside the housing,
   cooperating elements of the housing and the drug container holder capable, when engaged, of retaining the drug container holder in the end of injection position, and when disengaged, of releasing the biasing member,
wherein the safety device comprises a sheath lock having a leg extending radially between the drug container holder and the housing, and capable, in an end of injection position of the sheath lock, of disengaging the cooperating elements.

In a non-limiting embodiment, the plunger rod comprises a sheath lock contact surface configured for engaging with a plunger rod engaging area of the sheath lock to push axially the sheath lock in the proximal direction from a prior to injection position to the end of injection position of the sheath lock.

In a non-limiting embodiment, the cooperating elements comprise a retraction stop snap fit of the housing having the shape of a finger extending towards the proximal end, and a prior use retraction stop of the drug container holder. In the end of injection position of the drug container holder, the retraction stop snap fit bends radially inwards and has a proximally facing surface axially biased against a distally facing surface of the prior use retraction stop.

In a non-limiting embodiment, a housing unlock surface formed by an outer surface of a distal end of the leg is capable, in the end of injection position of the sheath lock, of pushing the retraction stop snap fit radially outwards to disengage the cooperating elements.

In a non-limiting embodiment, the sheath lock has a distal tubular portion, a distal portion of the leg of the sheath lock extending axially along the distal tubular portion, the distal tubular portion being wrapped around a distal end of the drug container holder in a prior to injection position of the sheath lock. In a non-limiting embodiment, the safety device comprises cooperating tabs of the housing and the drug container holder is capable, when engaged, of retaining the drug container holder in its final retracted lock-out position.

In a non-limiting embodiment, the cooperating tabs comprise a lock-out snap fit of the housing having the shape of a finger extending distally, and a sheath lock connection snap fit of the drug container holder. In the final retracted lock-out position of the drug container holder, the lock-out snap fit bends radially inwards and has a distally facing surface axially biased against a proximally facing surface of the sheath lock connection snap fit.

In a non-limiting embodiment, the cooperating tabs comprise a sheath lock connection snap fit of the drug container holder, the sheath lock comprising a support tongue, and in the final retracted lock-out position of the sheath lock, the support tongue is radially located between the sheath lock connection snap fit and the barrel of the drug container.

In a non-limiting embodiment, the cooperating tabs comprise a sheath lock connection snap fit of the drug container holder, the sheath lock connection snap fit having the shape of a finger extending in a distal direction and being configured to engage with stop ledges of the sheath lock in a prior to injection position of the sheath lock.

In a non-limiting embodiment, the sheath lock comprises a plunger rod snap fit engagement ring radially in contact with the plunger rod, the plunger rod snap fit engagement ring being, in a prior to injection position of the plunger rod, engaged with transport locks on the plunger rod.

In a non-limiting embodiment, the plunger assembly comprises an end of injection snap fit shelf located at a distal extremity of the plunger rod, and the drug container holder comprises plunger rod snaps configured to be snapped over the end of injection snap fit shelf in an end of injection position of the plunger rod.

In a non-limiting embodiment, the plunger assembly comprises a plunger head and a plunger neck, the plunger neck having a shape that is flared from the end of injection snap fit shelf towards of the plunger head, and in the end of injection position of the plunger rod, a distal extremity of the drug container holder abut axially the plunger neck.

In a non-limiting embodiment, the housing comprises a needle assembly stop element configured to engage with a proximal extremity of an arm of the needle assembly in the end of injection position of the drug container holder. In the present disclosure, when the term “distal direction” is used, this refers to the direction pointing away from the dose delivery site during use of the safety device. When the term “distal part/end” is used, this refers to the part/end of the safety device, or the parts/ends of the members thereof, which during use of the safety device is/are located furthest away from the dose delivery site. Correspondingly, when the term “proximal direction” is used, this refers to the direction pointing towards the dose delivery site during use of the safety device. When the term “proximal part/end” is used, this refers to the part/end of the safety device, or the parts/ends of the members thereof, which during use of the safety device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

When a component is said to move proximally, resp. distally, it should be understood that it moves towards a proximal end, resp. distal end, of the device. When a component is said to move proximally, distally, axially in a proximal direction, or axially in a distal direction, the movement is by default relative to the housing of the injection device, unless mentioned otherwise.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIG. 1A shows an injection device comprising a safety device according to a non-limiting embodiment of the invention, in a prior to injection configuration.

FIG. 1B shows a partial cross-section view of the injection device of FIG. 1A.

FIG. 2A shows a housing of the safety device of FIG. 1A

FIG. 2B shows internal features of the housing of FIG. 2A,

FIGS. 3A and 3B show a plunger assembly of the safety device of FIG. 1A.

FIG. 4 shows a drug container holder of the safety device of FIG. 1A.

FIGS. 5A and 5B show a sheath lock of the safety device of FIG. 1A.

FIG. 6A shows the injection device of FIG. 1A, in the prior to injection configuration, the housing being not shown and a cap member being see-through.

FIG. 6B shows a partial cross-section view of the injection device of FIG. 1A, in the prior to injection configuration.

FIG. 6C shows a cross-section view of a portion of the injection device of FIG. 1A, prior to injection.

FIG. 7A shows a partial cross-section view of the injection device of FIG. 1A, in an end of injection configuration.

FIG. 7B shows a partial cross-section view of a proximal part of the injection device of FIG. 1A in, in the end of injection configuration.

FIG. 7C shows a distal part of the injection device of FIG. 1A, in the end of injection configuration, the housing not being shown.

FIG. 8A shows a partial cross-section view of the injection device of FIG. 1A, in a final retracted lock-out configuration.

FIG. 8B shows a distal part of the injection device of FIG. 1A, in the final retracted lock-out configuration.

FIG. 8C is a cross-section view of a portion of the injection device of FIG. 1A, in the final retracted lock-out configuration.

FIGS. 9A, 9B, 9C and 9D show partial cross-section views of the injection device of FIG. 1A, in the packaged stage, prior to injection with a cap member off, at the end of the injection, and in the final retracted lock-out configuration.

FIG. 10A shows a sub-assembly, before final assembly, of a safety device according to an alternative non-limiting embodiment of the invention.

FIG. 10B shows the subassembly of FIG. 10A at a final assembly stage.

### DETAILED DESCRIPTION

To provide an overall understanding of the safety device described herein, certain illustrative embodiments will now be described.

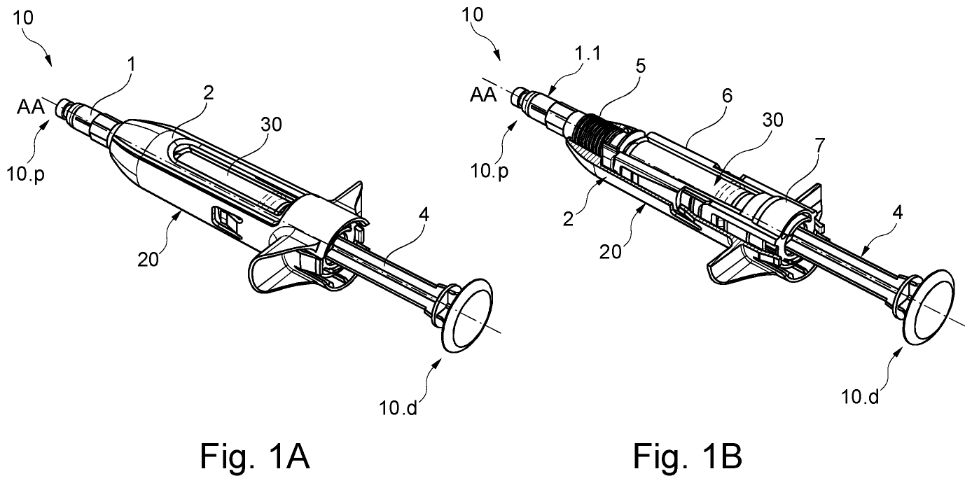

FIGS. 1A and 1B show an injection device 10 in a packaged state as it would be presented to a user, in a prior to injection configuration. FIG. 1B shows a partial cross-section view of the injection device 10.

The injection device 10 comprises a safety device 20 and a drug container 30, for instance a cartridge.

The injection device 10 extends along a longitudinal axis AA between a proximal end 10.p, where the drug is ejected, and a distal end 10.d. The proximal end 10.p is configured to point towards a dose delivery site (i.e., a patient's skin part) during use of the injection device 10, and a distal end 10.d pointing away from the dose delivery site.

The drug container 30 contains a set dosage of a drug.

The safety device 20 comprises a needle assembly 1, a housing 2, a plunger assembly 4, a biasing member 5, a drug container holder 6 and a sheath lock 7.

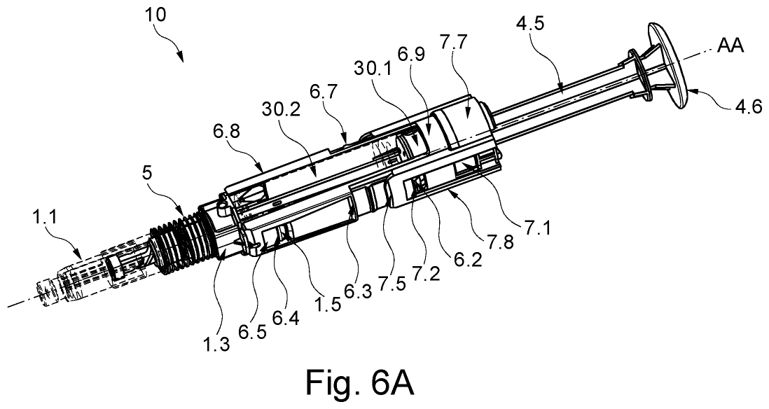

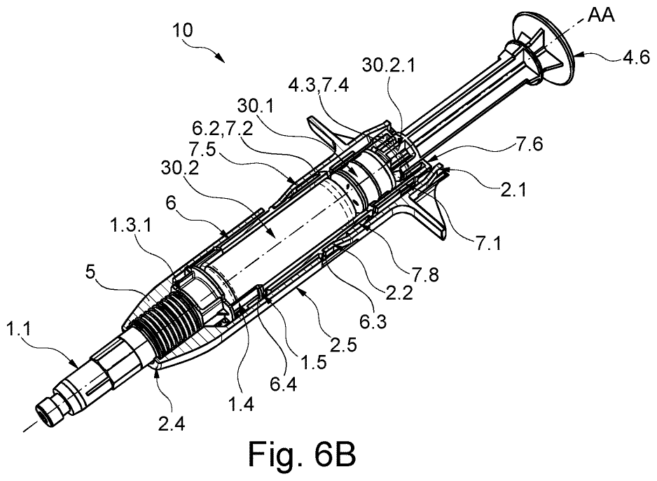

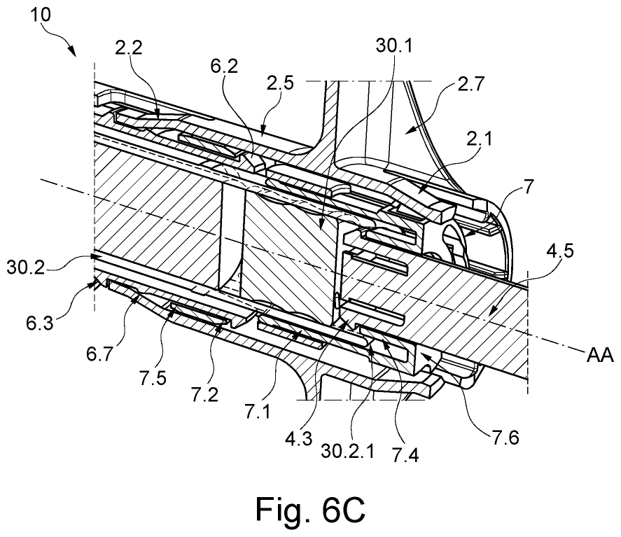

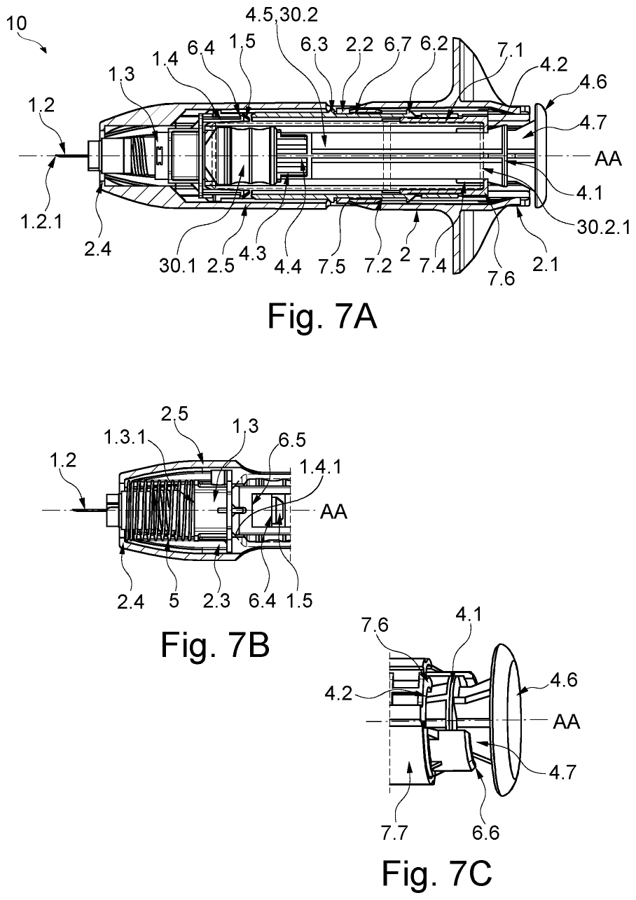

The needle assembly 1 of the safety device 20 is described hereafter with reference to FIGS. 6B and 7A.

The needle assembly 1 comprises a cap member 1.1, a needle 1.2, a retainer member 1.3, arms 1.4, hooks 1.5, and a hub 1.6.

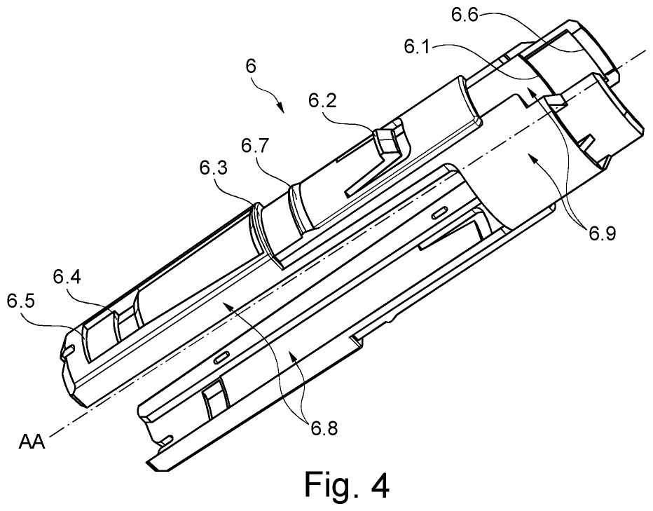

The cap member 1.1 is configured to cover the needle 1.2 of the needle assembly 1. Thus, the cap member 1.1 is removably attached to the rest of the needle assembly 1. When the cap member 1.1 is removed from the rest of the needle assembly 1, the needle 1.2 is exposed. The hub 1.6 holds the needle 1.2. The retainer member 1.3 ends with two arms 1.4 diametrically opposite one another. Another number of arms 1.4 is possible. The arms 1.4 extend from the hub 1.6, parallel to the longitudinal axis AA, in the distal direction. Each arm 1.4 ends with a hook 1.5. The hooks 1.5 are configured to be connected to the drug container holder 6 via an engagement between the hooks 1.5 and needle assembly snap fit ledges 6.4 of the drug container holder 6. The needle assembly snap fit ledges 6.4 of the drug container holder 6 are visible in FIG. 4, and the engagement between the hooks 1.5 and the needle assembly snap fit ledges 6.4 is shown in FIG. 6.

The cap member 1.1 is configured to be interactively connected to both the hub 1.6 and the retainer member 1.3. The needle 1.2 has a proximal pointed end 1.2.1 and distal pointed end. The distal pointed end of the needle 1.2 is configured to penetrate a membrane arranged at a proximal end of the drug container 30 when the cap member 1.1 is removed.

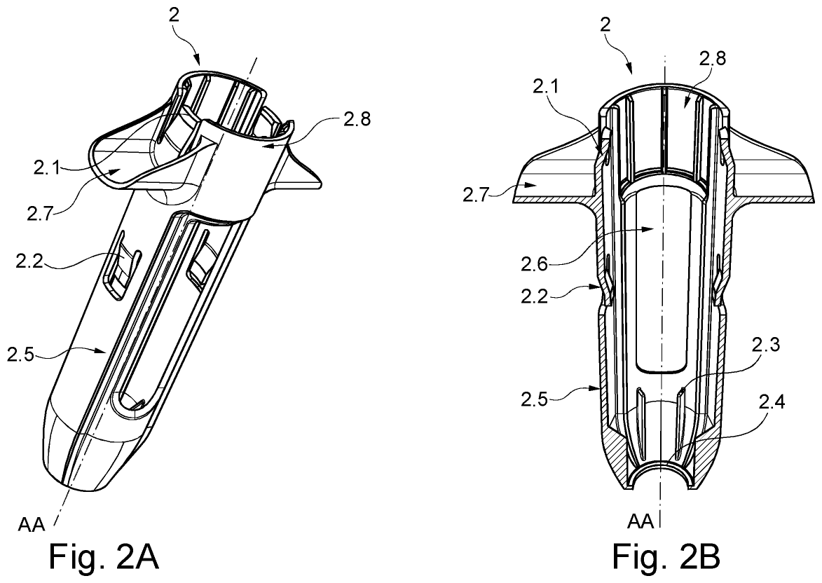

FIG. 2A shows the housing 2 of the safety device 20 in more detail; FIG. 2B shows internal features of the housing 2.

The housing 2 comprises two lock-out snap fits 2.1, two retraction stop snap fits 2.2, several needle assembly stop elements 2.3, a biasing member seat 2.4 at a proximal extremity, a case 2.5, a window 2.6, two handles 2.7 and a discontinuous annular portion 2.8 at a distal extremity.

The case 2.5 is generally tubular, but other shapes are possible. The case 2.5 extends along the longitudinal axis AA. The case 2.5 is hollow and defines an outer chamber. The outer chamber is dimensioned to receive the drug container holder 6, itself dimensioned to receive and hold the drug container 30. The case 2.5 comprises the optional window 2.6 through which a user can see the drug to be injected. The housing 2 further comprises the two diametrically opposite handles 2.7, extending from the case 2.5 orthogonally to the longitudinal axis AA. Another number of handles or no handles at all is possible. The discontinuous annular portion 2.8 extends from the case 2.5 in the distal direction around the longitudinal axis AA. The discontinuous annular portion 2.8 comprises two cut-outs in diametrically opposite positions, extending between the distal extremity of the housing 2 and the case 2.5.

The lock-out snap fits 2.1 are in diametrically opposite positions on the case 2.5, located in the cut-outs of the discontinuous annular portion 2.8. Another number of lock-out snap fits 2.1 is possible. Each lock-out snap fit 2.1 has the shape of a finger extending from the case 2.5 in the distal direction. The lock-out snap fits 2.1 bend radially inwards in a rest position and can flex radially outwards if a force is applied to them. The function of the lock-out snap fits 2.1 will be explained later.

The retraction stop snap fits 2.2 are in diametrically opposite positions on the case 2.5, located in cut-outs of the case 2.5, axially located roughly halfway the length of the case 2.5, Another number of retraction stop snap fits 2.2 is possible. Each retraction stop snap fit 2.2 has the shape of a finger extending from the case 2.5 in a proximal direction. The retraction stop snap fits 2.2 bend radially inwards in a rest position and can flex radially outwards if a force is applied to them. The retraction stop snap fits 2.2 are configured to cooperate with prior use retraction stops 6.3 of the drug container holder 6, in order to prevent distal movements of the needle assembly 1 and of the drug container holder 6 in the prior to injection configuration. The prior use retraction stops 6.3 of the drug container holder 6 are visible in FIG. 4, and the engagement between the retraction stop snap fits 2.2 and the prior use retraction stops 6.3 is visible in FIG. 6B. The prior use retraction stops 6.3 and the retraction stop snap fits 2.2 are called cooperating elements.

The needle assembly stop elements 2.3 extend axially from an inner surface of the case 2.5 and are configured to stop an axial movement of the needle assembly 1 in the proximal direction by contact with proximal extremities 1.4.1 of the needle assembly arms 1.4, as visible in FIG. 7B.

The biasing member seat 2.4 extends annularly orthogonally to the longitudinal axis AA at the proximal extremity of the case 2.5. The biasing member 5 is configured to be positioned between the biasing member seat 2.4 and a proximally facing surface 1.3.1 of the retainer member 1.3, as visible in FIG. 7B.

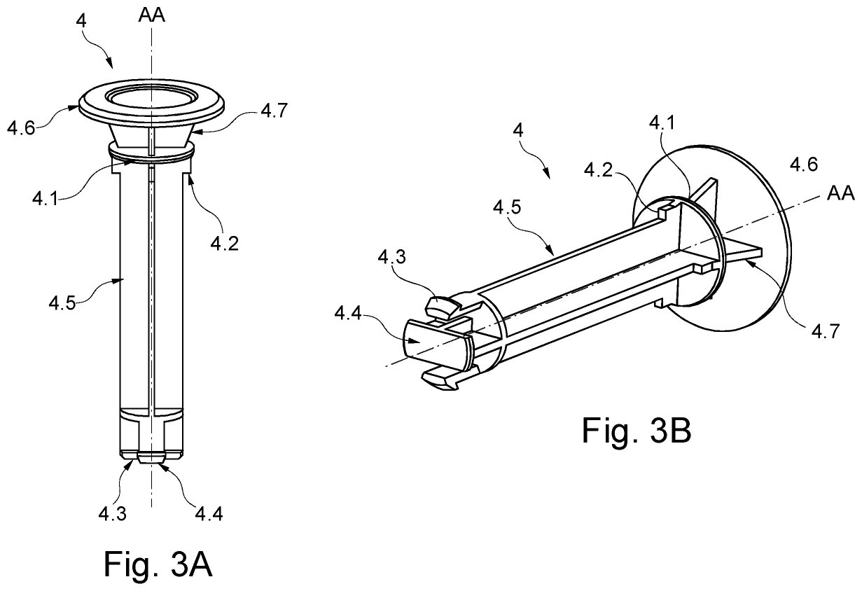

FIGS. 3A and 3B show the plunger assembly 4 of the safety device 20 in more detail.

The plunger assembly 4 of the safety device 20 includes an end of injection snap fit shelf 4.1, a sheath lock contact surface 4.2, two transport locks 4.3, a plunger contact surface 4.4, a plunger rod 4.5, a plunger head 4.6 and a plunger neck 4.7.

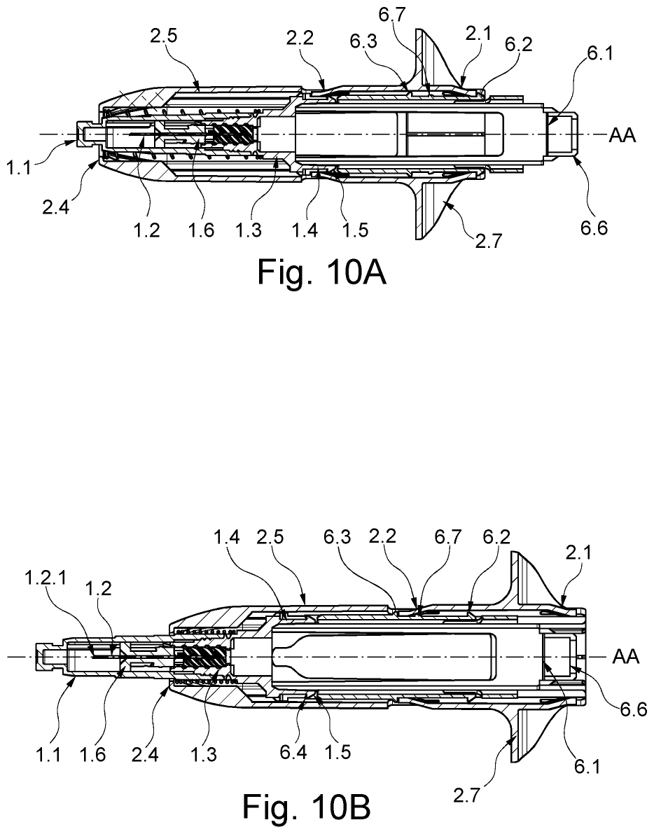

The plunger rod 4.5 extends substantially along the longitudinal axis AA. The plunger rod 4.5 is capable, during a displacement from a prior to injection position (shown in FIGS. 10A and 10B) to an end of injection position (shown in FIG. 10C) along the longitudinal axis AA, of pushing a plunger 30.1 of the drug container 30 inside a barrel 30.2 of the drug container 30, towards the proximal end 10.p, for expelling a dose of drug through the needle 1.2. The plunger 30.1 and the barrel 30.2 are both visible in FIGS. 6B and 6C. The plunger 30.1 is configured to contact the plunger rod 4.5 at the plunger contact surface 4.4. The plunger contact surface 4.4 extends substantially orthogonally to the longitudinal axis AA.

The plunger head 4.6 (also known as thumb rest) is formed at a distal extremity of the plunger assembly 4. The plunger head 4.6 is substantially annular and extends orthogonally to the longitudinal axis AA. Other shapes for the plunger head 4.6 are possible. The plunger neck 4.7 links the plunger head 4.6 to a distal extremity of the plunger rod 4.5. The plunger neck 4.7 has a shape that is flared towards the plunger head 4.6. The plunger head 4.6 is supposed to be pushed manually in the proximal direction, in order to move the plunger rod 4.5 from the prior to injection position to the end of injection position.

The end of injection snap fit shelf 4.1 is located at the distal extremity of the plunger rod 4.5, i.e., a proximal extremity of the plunger neck 4.7. The end of injection snap fit shelf 4.1 is substantially disk-shaped. The end of injection snap fits shelf 4.1 is configured to cooperate with plunger rod snaps 6.6 on a distal end of the drug container holder 6. During injection, pushing the plunger head 4.6 in the proximal direction is only possible until the end of injection snap fit shelf 4.1 passes the plunger rod snaps 6.6 and the plunger rod snaps 6.6 abut axially the plunger neck 4.7. That position, shown in FIG. 7C, corresponds to the plunger rod 4.5 end of injection position.

To deliver an injection using the injection device 10, a user can grab the handles 2.7 of the housing 2, remove the cap member 1.1 from the needle assembly 1, insert the needle 1.2 into a delivery site, and deliver the injection by pressing the plunger head 4.6 of the plunger assembly 4 towards the housing 2, that is to say in the direction of the proximal end 10.p, until the plunger rod 4.5 reaches the end of injection position.

The sheath lock contact surface 4.2 is a contact surface for a plunger rod engaging area 7.6 of the sheath lock 7, shown in FIG. 7A. The sheath lock contact surface 4.2 is located on a distal part of the plunger rod 4.5, a few millimetres away in the proximal direction from the end of injection snap fit shelf 4.1. The sheath lock contact surface 4.2 extends orthogonally to the longitudinal axis AA.

The transport locks 4.3 are located on either side of the plunger contact surface 4.4, at a proximal side of the plunger rod 4.5. The transport locks 4.3 are configured to engage with a plunger rod snap fit engagement ring 7.4 of the sheath lock 7 during transportation, as shown in FIGS. 6B and 6C, in order to avoid distal movements of the plunger rod 4.5.

FIG. 4 shows the drug container holder 6 of the safety device 20 in more detail.

The drug container 6 comprises two drug container support ledges 6.1, two sheath lock connection snap fits 6.2, the two prior use retraction stops 6.3, the two needle assembly snap fit ledges 6.4, two after use retraction stops 6.5, the two plunger rod snaps 6.6, two slopes 6.7, two legs 6.8, two arms 6.9.

The drug container holder 6 extends generally along the longitudinal axis AA. The legs 6.8 extend axially and are diametrically opposite one another. The arms 6.9 extend axially and are diametrically opposite one another too, but in different angular sections compared to the legs 6.8. The legs 6.8 are linked to each other, at their distal side, by a proximal side of the arms 6.9, and the arms 6.9 are linked to each other, at their proximal side, by a distal side of the legs 6.8. It should be noted that because of the chosen partial cross-section views of FIGS. 6B, 7A and 8A, the arms 6.9 are not visible in those figures.

The drug container holder 6 is configured to hold the drug container 30 within the housing 2. The drug container 30 comprises the aforementioned barrel 30.2, generally cylindrical although other shapes are possible, the aforementioned plunger 30.1, located inside the barrel 30.2 and configured to be axially pushed by the plunger rod 4.5 until it reaches a neck at a proximal end of the barrel 30.2, and a membrane closing the neck. The barrel 30.2 comprises a distal extremity 30.2.1 defining an opening for the plunger rod 4.5, visible for instance in FIGS. 6B, 6C and 7A.

The drug container support ledges 6.1 are each located on the inner surface of an arm 6.9 of the drug container 6. Each drug container support ledge 6.1 extends orthogonally to the longitudinal axis AA and is configured for supporting the distal extremity 30.2.1 of the barrel 30.2.

The sheath lock connection snap fits 6.2 are each located in a cut-out of a leg 6.8 of the drug container 6. Each sheath lock connection snap fit 6.2 has the shape of a finger extending from the leg 6.8 in a distal direction. The sheath lock connection snap fits 6.2 are configured to engage with stop ledges 7.2 of the sheath lock via a snap fit attachment in prior to injection positions of the drug container holder 6 and the sheath lock 7. The sheath lock connection snap fits 6.2 are configured to engage with the lock-out snap fits 2.1 of the housing 2 in a final retracted lock-out position of the drug container holder 6. It should be noted that the sheath lock connection snap fits 6.2 are flexible radially inwards for assembly purposes.

The prior use retraction stops 6.3 are each located on the outer surface of a leg 6.8 of the drug container holder 6. The prior use retraction stops 6.3 are configured to cooperate with the retraction stop snap fits 2.2 of the housing 2, in order to prevent distal movements of the needle assembly 1 and the drug container holder 6 in the prior to injection configuration. The engagement between the prior use retraction stops 6.3 and the retraction stop snap fits 2.2 is visible in FIG. 6B, FIG. 10A and FIG. 10B. Each prior use retraction stop 6.3 has the shape of a ledge extending orthogonally to the longitudinal axis AA along the width of the leg 6.8 and configured for contacting a proximally facing surface of a retraction stop snap fit 2.2 of the housing 2.

The needle assembly snap fit ledges 6.4 are each located on a leg 6.8 of the drug container holder 6. The needle assembly snap fit ledges 6.4 are configured to connect the drug container holder 6 to the needle assembly 1, via an engagement with the hooks 1.5 of the retainer member 1.3 of the needle assembly 1. The engagement between the hooks 1.5 and the needle assembly snap fit ledges 6.4 is shown in FIG. 6A.

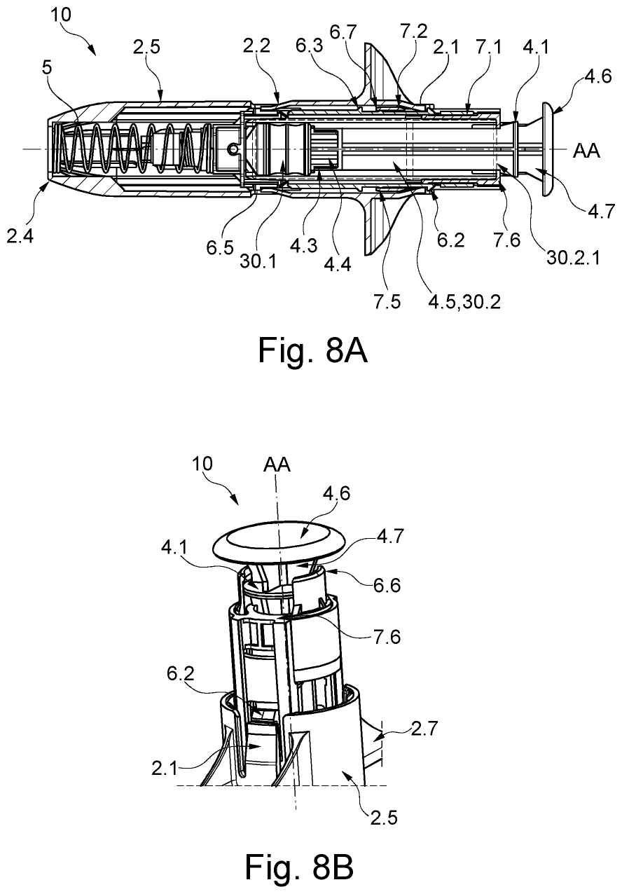

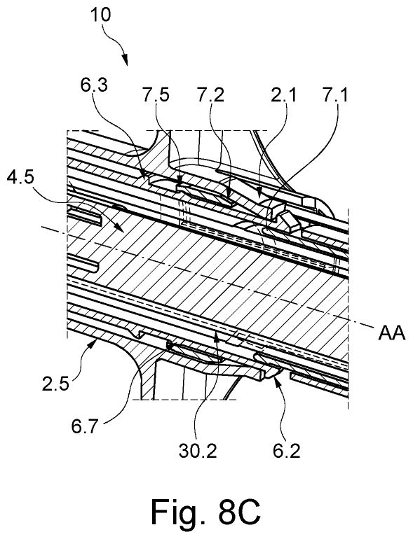

The after use retraction stops 6.5 are each located on the outer surface of a leg 6.8 of the drug container holder 6. The after use retraction stops 6.5 are configured to cooperate with the retraction stop snap fits 2.2 of the housing 2, in order to prevent distal movements of the needle assembly 1 and the drug container holder 6 in the final retracted lock-out configuration. The engagement between the after use retraction stops 6.5 and the retraction stop snap fits 2.2 is visible in FIG. 8A. Each after use retraction stop 6.5 has the shape of a ledge extending orthogonally to the longitudinal axis AA and configured for contacting a proximally facing surface of a retraction stop snap fit 2.2 of the housing 2.

The plunger rod snaps 6.6 are each located at the distal ends of the arms 6.9 of the drug container holder 6. The plunger rod snaps 6.6 extend radially inwards, orthogonally to the longitudinal axis AA. During injection, pushing the plunger head 4.6 in the proximal direction is only possible until the end of injection snap fit shelf 4.1 of the plunger assembly 4 passes the plunger rod snaps 6.6 and the plunger rod snaps 6.6 abut axially the plunger neck 4.7.

That position, shown in FIGS. 7A and 7C corresponds to the plunger rod 4.5 end of injection position.

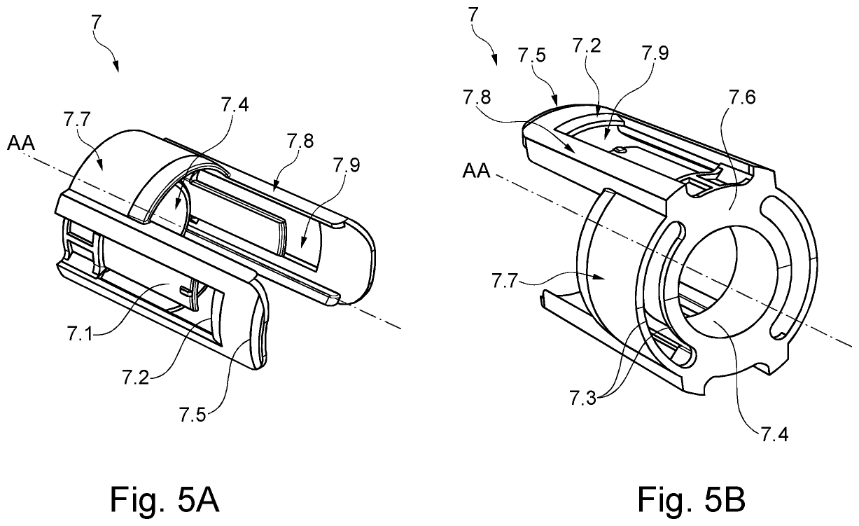

The slopes 6.7 are each located on the outer surface of the arms 6.9 of the drug container holder 6. Each slope 6.7 is located on an arm 6.9 between the sheath lock connection snap fit 6.2 and the prior use retraction stop 6.3. Each slope 6.7 has the shape of a ledge extending orthogonally to the longitudinal axis AA along the width of the arm 6.9. Their function will be explained later. FIGS. 5A and 5B show the sheath lock 7 of the safety device 20 in more details. The sheath lock 7 comprises two support tongues 7.1, the two stop ledges 7.2, two drug container holder tracks 7.3, the plunger rod snap fit engagement ring 7.4, two housing unlock surfaces 7.5, the plunger rod engaging area 7.6, a distal tubular portion 7.7, and two legs 7.8.

The plunger rod snap fit engagement ring 7.4 and the distal tubular portion 7.7 extend concentrically with each other along the longitudinal axis AA from a distal end of the sheath lock 7. The plunger rod snap fit engagement ring 7.4 extends closer to the longitudinal axis AA than the distal tubular portion 7.7 and has a shorter axial width.

The plunger rod snap fit engagement ring 7.4 and the distal tubular portion 7.7 are linked to each other at the distal end of the sheath lock 7 by the plunger rod engaging area 7.6, which extends orthogonally to the longitudinal axis AA. The plunger rod engaging area 7.6 comprises 3 through openings: a central opening defined by the plunger rod snap fit engagement ring 7.4, and two diametrically opposite circular arcs located between the distal tubular portion 7.7 and the plunger rod snap fit engagement ring 7.4, those circular arcs being the drug container holder tracks 7.3.

The legs 7.8 extend axially in the proximal direction from the distal end of the sheath lock 7. The legs 7.8 are diametrically opposite one another and linked to each other by the parts of the plunger rod engaging area 7.6 located between the extremities of the drug container holder tracks 7.3. The axial length of the legs is higher than the axial width of the distal tubular portion 7.7, and a distal portion of each of the legs 7.8 extends axially along the distal tubular portion 7.7. The housing unlock surfaces 7.5 are the outer surfaces of the distal ends of the legs 7.8.

The support tongues 7.1 are diametrically opposite one another and extend axially in the proximal direction from the parts of the plunger rod engaging area 7.6 located between the extremities of the drug container holder tracks 7.3. Each leg 7.8 comprises a through cut-out 7.9. The support tongues 7.1 are located at the same angular position as the cut-outs 7.9 but are closer to the longitudinal axis AA than the legs 7.8. The support tongues 7.1 are radially located between the barrel 30.2 of the drug container 30 and the drug container holder 6. The stop ledges 7.2 are the proximal edges of the cut-outs 7.9.

FIGS. 6A, 6B and 6C show the injection device 10 in the prior to injection configuration. In FIG. 6A, the housing 2 is not shown and the cap member 1.1 is see-through 1.1. FIG. 6B shows a partial cross-section view of the injection device 10. FIG. 6C shows a cross-section of a portion of the injection device 10.

In the prior to injection configuration, the biasing member 5 is in a compressed state and the plunger rod 4.5 is in the prior to injection position. As visible in FIG. 6A, prior to injection, the distal tubular portion 7.7 of the sheath lock 7 is placed around the distal end of the drug container holder 6. Thus, the sheath lock 7 prevents the drug container holder 6 from flexing radially outwards, thereby axially securing or locking the drug container 30 inside the drug container holder 6. The legs 7.8 of the sheath lock 7 extend against the drug container holder 6.

As visible in FIG. 6B and more clearly in FIG. 6C, the plunger rod snap fit engagement ring 7.4 of the sheath lock 7 is engaged with the transport locks 4.3 of the plunger assembly 4 so that a distal movement of the plunger rod 4.5 relative to the sheath lock 7 is not possible. Besides that, the sheath lock connection snap fits 6.2 of the drug container holder 6 are engaged with the stop ledges 7.2 of the sheath lock 7 so that a proximal movement of the drug container holder 6 relative to the housing 2 would lead to a proximal movement of the sheath lock 7. Finally, the retraction stop snap fits 2.2 of the housing 2 are engaged with the prior use retraction stops 6.3 of the drug container holder 6 so that distal movements of the needle assembly 1 and the drug container holder 6 relative to the housing 2 are not possible.

FIGS. 7A, 7B and 7C show the injection device 10 in an end of injection configuration. FIG. 7A shows a partial cross-section view of the injection device 10. FIG. 7B shows a partial cross-section view of a proximal part of the injection device 10. FIG. 7C shows a distal part of the injection 10 device, the housing 2 not being shown.

### The biasing member 5 is still in a compressed state, but the plunger rod 4.5 is in the end of injection position.

As visible in FIG. 7A, the drug container holder 6 has not moved axially relative to the housing 2. However, the sheath lock 7 has moved proximally relative to the housing 2 and the drug container holder 6. In FIG. 6B, the sheath lock 7 was in a prior to injection position; in FIG. 7A the sheath lock is in an end of injection position. The sheath lock 7 has moved proximally because during the axial movement of the plunger rod 4.5 from the prior to injection position to the end of injection position, the sheath lock contact surface 4.2 of the plunger assembly 4 has come into contact with the plunger rod engaging area 7.6, which has pushed proximally the sheath lock 7.

In the end of injection configuration, the plunger rod snap fit engagement ring 7.4 of the sheath lock 7 is inside the distal end of the barrel 30.2 of the drug container 30, and the plunger rod snap fit engagement ring 7.4 is radially biased against a distal portion of the plunger rod 4.5.

In the end of injection configuration, the housing unlock surfaces 7.5 of the sheath lock 7 are pushing the retraction stop snap fits 2.2 of the housing radially outwards. The retraction stop snap fits 2.2 of the housing are about to be pushed radially outwards enough to disengage with the prior use retraction stops 6.3 of the drug container holder 6. Besides that, the inner surfaces of the distal ends of the legs 7.8 are biased against the slopes 6.7 on the outer surface of the drug container holder 6.

As visible in FIG. 7B, the needle assembly stop elements 2.3 of the housing 2 are, during injection, in contact with the proximal extremities 1.4.1 of the needle assembly arms 1.4, thereby preventing the needle assembly 1 from moving in the proximal direction.

As visible in FIG. 7C, the end of injection snap fits shelf 4.1 has passed the plunger rod snaps 6.6 of the drug container holder 6, preventing the plunger rod 4.5 from moving distally relative to the drug container holder 6.

FIGS. 8A, 8B and 8C show the injection device 10 in a final retracted lock-out configuration. FIG. 8A shows a partial cross-section view of the injection device 10; FIG. 8B shows a distal part of the injection device 10, FIG. 8C shows a cross-section of a portion of the injection device 10.

In the final retracted lock-out configuration, the biasing member 5 is in a released state, also called extended state. The needle assembly 1, the drug container holder 6, the sheath lock 7 and the plunger assembly 4 have moved distally and are in final retracted lock-out positions.

The releasing of the biasing member 5 has been made possible by the disengagement of the retraction stop snap fits 2.2 of the housing with the prior use retraction stops 6.3 of the drug container holder 6, making it possible for the drug container holder 6 to move distally the moment the user removes the force applied to the plunger head 4.6. The biasing member has in turned pushed distally the needle assembly 1 and thus the drug container 30 and the drug container holder 6. The distal movement of the distal end 30.2.1 of the drug container 30, has in addition also pushed the sheath lock 7 in the distal direction. Since the sheath lock contact surface 4.2 of the plunger assembly 4 is in contact with the plunger rod engaging area 7.6 of the sheath lock 7-the plunger rod assembly 4 also moves distally.

It should be noted that the proximal movement of the plunger assembly 4 stops in the end of injection configuration when the proximal movement of sheath lock 7 stops at the distal end 30.2.1 of the drug container 30, as shown in FIG. 7A. Consequently, a small volume of the drug might remain in the barrel 30.2. In this configuration, the releasing of the biasing member 5 will result in that the plunger rod engaging area 7.6 of the sheath lock 7 will, during lock out retraction, push the plunger assembly 4 distally to the position shown in FIG. 8A.

In an alternative end of injection configuration, the proximal movement of the plunger rod assembly 4 stops when the plunger 30.1-pushed by the plunger contact surface 4.4, reaches its proximal end position by the neck of the barrel 30.2. Consequently, all the drug contained in the barrel 30.2 will be expelled. In this configuration the releasing of the biasing member 5 will result in that during lock out retraction, the plunger assembly 4 will be pushed distally by the plunger 30.1 to the position shown in FIG. 8A.

In the final retracted lock-out configuration, the after use retraction stop 6.5 of the drug container holder 6 engage with the retraction stop snap fit 2.2 of the housing 2, preventing further distal movement of the drug container holder 6, and consequently of the needle assembly 1, sheath lock 7 and plunger assembly 4.

Between the end of injection position and the final retracted lock-out position of the sheath lock 7, the lock-out snap fits 2.1 of the housing 2 flex radially outwards and then return to their radially inwardly flexed resting position to engage with the sheath lock connection snap fit 6.2 of the drug container holder 6 in the final retracted lock-out position of the sheath lock 7, as visible in FIG. 8C. The lock-out snap fits 2.1 of the housing 2 and the sheath lock connection snap fit 6.2 of the drug container holder 6 are called cooperating tabs.

In the final retracted lock-out position of the sheath lock 7, the support tongue 7.1 of the sheath lock 7 restricts the movement of the sheath lock connection snap fits 6.2, preventing the sheath lock connection snap fits 6.2 to flex radially inwards and to lose their connection to the lock-out snap fits 2.1.

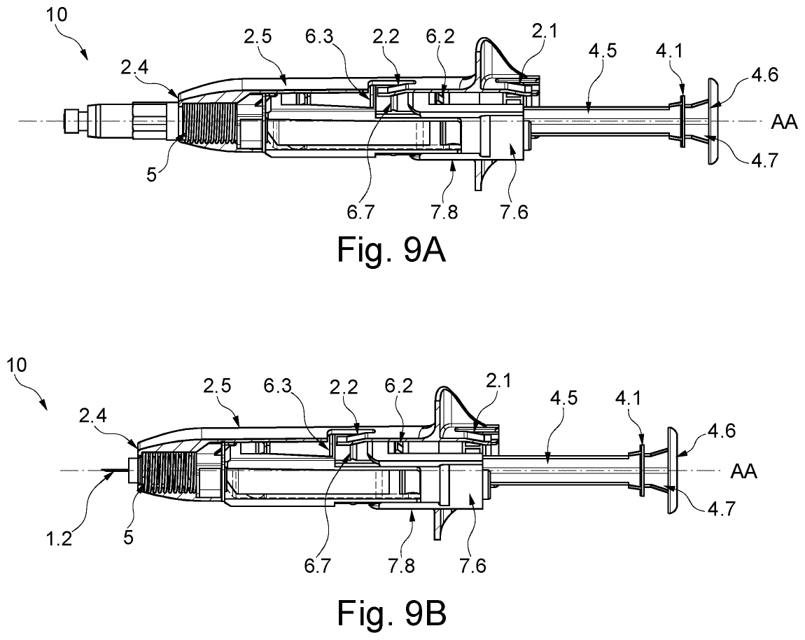

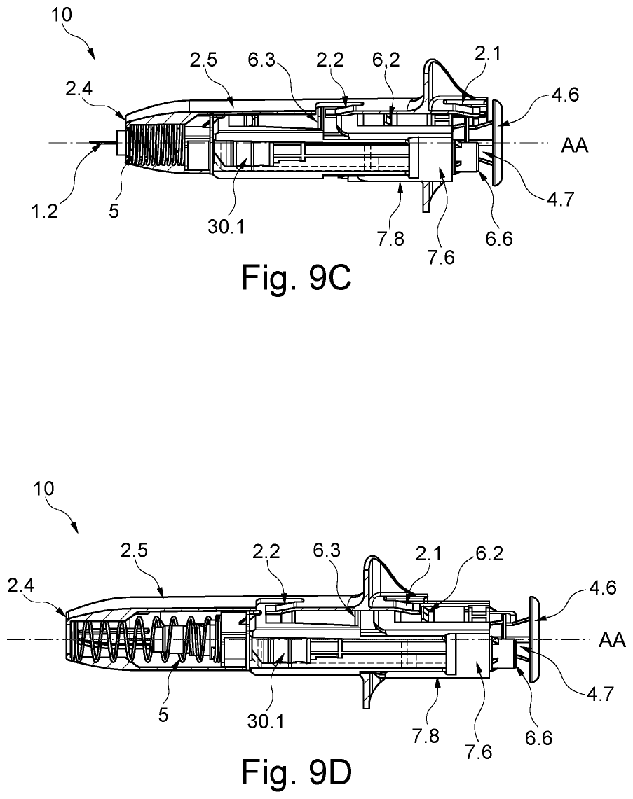

FIGS. 9A, 9B, 9C and 9D are partial cross-section views of the injection device, in the packaged stage, prior to injection with the cap member 1.1 off, in the end of injection configuration, and in the final retracted lock-out configuration.

As can be understood, the invention uses an axially moving sheath lock 7 that is moved at the end of the injection stroke. The sheath lock 7 unlocks the retraction stop snap fits 2.2 on the housing that were engaged with the prior use retraction stops 6.3 of the drug container holder 6, thereby releasing the pre-compressed biasing member 5 in order to initiate needle retraction and lock out. The needle assembly 1 and the drug container 30 are both pushed in the distal direction relative to the housing 2 after the releasing of the biasing member 5. Then the drug container holder 6 ends up at its final retracted lock-out position, and the lock-out snap fits 2.1 of the housing 2 engage with the sheath lock connection snap fit 6.2 of the drug container holder 6, preventing the needle from being accessed by the patient.

It should be noted that both the priming sequence and injection are fully manual, the only automated function is the lock-out of the needle once the injection is finished and the user has removed pressure from the plunger rod head 4.6.

FIG. 10A shows a sub-assembly, before final assembly, of a safety device according to an alternative non-limiting embodiment of the invention. FIG. 10B shows said subassembly in a final assembly configuration.

In the alternative embodiment, before the final assembly, the biasing member 5 is only lightly loaded. This releases stress on plastic components, which is useful during the transportation and storage periods. The needle assembly 1 and the drug container holder 6 are locked on the housing 2, preventing proximal and distal movements relative to the housing 2.

As visible in FIG. 10A, before the final assembly, the biasing member 5 is less compressed than in the previously described embodiment. The retraction stop snap fits 2.2 of the housing are engaged with the after use retraction stops 6.5 of the drug container holder 6, preventing distal movements of the needle assembly 1 and the drug container holder 6 relative to the housing 2. The lock-out snap fits 2.1 of the housing are engaged with the sheath lock connection snap fits 6.2 of the drug container holder 6, preventing proximal movements of the needle assembly and the drug container holder 6.

As visible in FIG. 10B, the final assembly is achieved by pushing the sheath lock connection snap fits 6.2 of the drug container holder 6 radially inwards so that they are releases from the lock-out snap fits 2.1 of the housing, and then push the drug container holder 61 in the proximal direction until the prior use retraction stops 6.3 of the drug container holder 6 are snapped to the retraction stop snap fits 2.2 of the housing 2.

The injection devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the injection devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the injection devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the injection devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDW137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies. Exemplary drugs that could be included in the injection devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the injection devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the injection devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the injection devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the injection devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the injection devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients. Exemplary drugs that could be included in the injection devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Various modifications to the embodiments described are possible and will occur to those skilled in the art without departing from the invention which is defined by the following claims.

1-13. (canceled)

14. An injection device for delivering a drug, extending generally along a longitudinal axis, and having a proximal end configured to point towards a dose delivery site and a distal end pointing away from the dose delivery site, the injection device comprising:
a drug container comprising a barrel and a plunger axially movable inside the barrel; and
a safety device comprising:
a housing;
a drug container holder holding the drug container in the housing;
a needle assembly attached to a proximal end of the drug container holder and comprising a needle;
a manually actuatable plunger assembly comprising a plunger rod capable of proximally pushing the plunger for expelling a dose of the drug through the needle;
a biasing member capable, when released, of moving the needle assembly and the drug container holder from an end of injection position where the needle extends partially out of a proximal part of the housing, to a final retracted lock-out position where the needle extends fully inside the housing; and
cooperating elements of the housing and the drug container holder is capable, when engaged, of retaining the drug container holder in the end of injection position, and when disengaged, of releasing the biasing member,
wherein the safety device comprises a sheath lock having a leg extending radially between the drug container holder and the housing, and capable, in an end of injection position of the sheath lock, of disengaging the cooperating elements.

15. The injection device according to claim 14, wherein the plunger rod comprises a sheath lock contact surface configured for engaging with a plunger rod engaging area of the sheath lock to push axially the sheath lock in the proximal direction from a prior to injection position to the end of injection position of the sheath lock.

16. The injection device according to claim 14,
wherein the cooperating elements comprise a retraction stop snap fit of the housing having the shape of a finger extending towards the proximal end, and a prior use retraction stop of the drug container holder,
wherein in the end of injection position of the drug container holder, the retraction stop snap fit bends radially inwards and has a proximally facing surface axially biased against a distally facing surface of the prior use retraction stop.

17. The injection device according to claim 16, wherein a housing unlock surface formed by an outer surface of a distal end of the leg is capable, in the end of injection position of the sheath lock, of pushing the retraction stop snap fit radially outwards to disengage the cooperating elements.

18. The injection device according to claim 14, wherein the sheath lock has a distal tubular portion, a distal portion of the leg of the sheath lock extending axially along the distal tubular portion, the distal tubular portion being wrapped around a distal end of the drug container holder in a prior to injection position of the sheath lock.

19. The injection device according to claim 14, wherein the safety device comprises cooperating tabs of the housing and the drug container holder is capable, when engaged, of retaining the drug container holder in its final retracted lock-out position.

20. The injection device according to claim 19, wherein the cooperating tabs comprise a lock-out snap fit of the housing having the shape of a finger extending distally, and a sheath lock connection snap fit of the drug container holder, wherein in the final retracted lock-out position of the drug container holder, the lock-out snap fit bends radially inwards and has a distally facing surface axially biased against a proximally facing surface of the sheath lock connection snap fit.

21. The injection device according to claim 20, wherein the cooperating tabs comprise a sheath lock connection snap fit of the drug container holder, the sheath lock comprising a support tongue, and wherein in the final retracted lock-out position of the sheath lock, the support tongue is radially located between the sheath lock connection snap fit and the barrel of the drug container.

22. The injection device according to claim 21, wherein the cooperating tabs comprise a sheath lock connection snap fit of the drug container holder, the sheath lock connection snap fit having the shape of a finger extending in a distal direction and being configured to engage with stop ledges of the sheath lock in a prior to injection position of the sheath lock.

23. The injection device according to claim 14, wherein the sheath lock comprises a plunger rod snap fit engagement ring radially in contact with the plunger rod, the plunger rod snap fit engagement ring being, in a prior to injection position of the plunger rod, engaged with transport locks on the plunger rod.

24. The injection device according to claim 14, wherein the plunger assembly comprises an end of injection snap fit shelf located at a distal extremity of the plunger rod, and the drug container holder comprises plunger rod snaps configured to be snapped over the end of injection snap fit shelf in an end of injection position of the plunger rod.

25. The injection device according to claim 24, wherein the plunger assembly comprises a plunger head and a plunger neck, the plunger neck having a shape that is flared from the end of injection snap fit shelf towards of the plunger head, and in the end of injection position of the plunger rod, a distal extremity of the drug container holder abut axially the plunger neck.

26. The injection device according to claim 14, wherein the housing comprises a needle assembly stop element configured to engage with a proximal extremity of an arm of the needle assembly in the end of injection position of the drug container holder.
